(19) United States
(12) Patent Application Publication
(10) Pub. No.: US 2002/0059937 A1
(43) Pub. Date: May 23, 2002

(54) Appliance for fixation and tension of knees under MRI examination

(76) Inventor: Peter C. Sullenberger, Shoreline, WA (US)

Correspondence Address: BLACK LOWE & GRAHAM, 816 SECOND AVE., SEATTLE, WA, 98104, US

(21) Appl. No.: 09/991,396

(22) Filed: Nov. 20, 2001

Related U.S. Application Data
(60) Provisional application No. 60/252,878, filed on Nov. 22, 2000.

(51) Int. Cl. A61F 5/37

(52) U.S. Cl. 128/846

(57) ABSTRACT

The device is a diagnostic tool for the determination of the functional loss of the soft tissue of one or both knees. The device allows for flexion of the knees against a biasing force for observation by magnetic resonance imaging. A supporting member is hingedly connected to a lever arm, both transparent to MRI. In turn, lower extremities of the knees under study are affixed to the lever arm. The lever arm is biased toward full extension by tension applied to the lever arm through a tensioning device. The tensioning device comprises elastic cords and several pulleys configured to apply a nearly constant biasing force throughout a range of flexion. Within the lever arm, an image generating device comprising a vial of electrolyte generates a bar of length proportionate to the degree of flexion.

### PRIORITY INFORMATION

This application claims priority to provisional application Ser. No. 60/252,878, filed Nov. 22, 2000.

### FIELD OF THE INVENTION

This invention relates generally to radiological examination and, more specifically, to appliances for joint fixation under radiological examination.

### BACKGROUND OF THE INVENTION

Knee injuries can be very painful and debilitating. Not all injuries to the knee joints or to the supporting ligaments and cartilage, however, require surgery; rest and physical therapy can allow the body to heal many ligament injuries. Success generally depends upon the residual health of the affected ligaments. Determining the state of health and the extent of the damage to ligaments allows the physician to evaluate whether or not surgery is required. The typical method for evaluating damage to ligaments is to view the knee under either magnetic resonance imaging (MRI) or x-ray radiography.

X-ray radiography is useful for imaging the hard tissue configuration but cannot yield more information as to the health of ligaments than might be inferred from hard tissue positioning. MRI can directly image the affected ligament. A single view of the ligament, especially in a relaxed posture, is unable to present sufficient information to allow the radiologist to assess function of the ligament.

Devices are known for stabilizing a limb of the body for examination. As for example, U.S. Pat. No. 5,136,743 to Pirela-Cruz discloses a device for positioning the distal radiohumal joint (DRUJ) for medical examination. The Pirela-Cruz device allows stress to be applied to the radius and ulna to assess stability in the DRUJ and is especially useful for a CAT scans or X-rays.

U.S. Pat. No. 5,163,443 to Fry-Welch et al measures the forces which are applied by a limb and is useful for determining the presence of cumulative trauma disorders, such as carpal tunnel syndrome.

U.S. Pat. No. 5,724,991, to Rijke, et al. issued on Mar. 10, 1998, and entitled “Wrist Fixation Device For Elbow Stress Examination” teaches a diagnostic tool for the determination of the functional loss of the collateral ligaments of the elbow. The device serves to position and to stabilize the arm of a subject and to position the elbow in a predetermined fixed position to allow for X-raying of elbow joints under graded pressure.

The foregoing patents illustrated that there is a need for various devices to position limbs during analysis of injuries, as well as techniques to evaluate the injury. None of the prior art have addressed the problem of examining bony structures with magnetic resonance imaging to reliably and reproducibly assessing injury to ligaments. In the instant invention, the extent of the injury can be evaluated non-invasively, particularly in the knee and proximate area.

### SUMMARY OF THE INVENTION

In one embodiment, the present invention is an appliance for the fixing of a joint under an imaging study. The appliance can both index the movement of the knee joint of a patient, the joint being movable into a particular orientation. While the current embodiment is directed toward the movement of the knee joint, any joint of the body can be studied with equal efficiency by simply reconfiguring the principal elements of the invention. One skilled in the art can readily see how the same principal elements will allow the study of an elbow, ankle or wrist in a range of movement.

The principal features of the appliance are an inexpensive, durable and reliable configuration of a lever arm to secure the distal member of the joint and to provide constant uniform force throughout the range of motion using elastics and pulleys. On that lever arm a free sliding leg plate accommodates variable patient size, and adjusts lever arm length to vary the applied force.

The free sliding leg plate carries a pair of inverted leg cradles in order to maximize comfort, range of motion and to minimize set up time. These leg cradles slide freely in the lateral direction in order to allow internal and external rotation of lower leg.

Within the lever arm, a “phantom window” or capsule indicator marks each MRI with a clear indication of position on the produced image. The radial distance through the window from the vertex of joint varies over each five-degree arc segment of movement. This imaging is accomplished by filling the phantom capsule with a solution of electrolytes that mimic body tissue in resonance. Thus, as the joint moves from zero degrees to forty-five degrees of flexion, the image of the “phantom capsule” appears as a column of electrolyte of varying lengths. In the preferred embodiment, this column varies in discrete steps rather than as a continuous function of position.

The present invention comprises a system for positioning a knee joint for examination by means of magnetic resonance imaging. The device operates by fixing the position of the knees in relation to a stationary base while allowing them to flex. A lever arm hingedly attached to the stationary base follows the flexion of the lower extremity of the knee. By cradling means, the lever arm is secured to lower extremity. By imparting a biasing force to the lever arm, a tensioning arm can place the examined knee under a biasing load. An inventive elastic tensioning member comprising blocks and loops of elastomeric cord imparts the biasing force.

In accordance with further aspects of the invention, a “phantom window”, a shaped capsule comprising an electrolytic solution generates a bar of length proportional to the degree of knee flexion when examined under MRI.

In accordance with other aspects of the invention, the tensioning device allows predictable bias loading of the knee by selectively engaging at least one of the plurality of elastic loops.

In accordance with still further aspects of the invention, the tension can be further adjusted by means of attachment of the tensioning device in one of the several attachment points within the stationary base.

As will be readily appreciated from the foregoing summary, the invention provides for studying the ligaments of a knee under tension. Imparting the tension to the knee joint may be by any of several means known in the art. The selection of cords of anhydrous elastomer is not meant to limit the invention where means exist that are known in the art to impart the tensioning force to the lever arm.

### BRIEF DESCRIPTION OF THE DRAWINGS

The preferred and alternative embodiments of the present invention are described in detail below with reference to the following drawings.

FIG. 1 is a perspective view of the inventive appliance;

FIG. 2 is a perspective view similar to that of FIG. 1 but including the imaging coils that may be positioned for detailed imaging;

FIGS. 3 and 4 show the block and bushing configuration of the variable tensioner and in FIG. 4 the placement of elasticized loops lending discrete variable tensions;

FIG. 5 shows the configuration of the tensioning arm with the variable tensioner;

FIG. 6 shows the cowling for support of the tensioning arm assembly;

FIGS. 7 and 8 portray the cowling assembly at full extension and at maximum flexion respectively;

FIGS. 9 and 10 portray the leverage configuration for constant tensioning through out the range of motion at full extension and at maximum flexion respectively;

FIG. 11 shows the operation of the “phantom window” flexion indicator; and

FIG. 12 is a graph of the imaging of the “phantom window” indicative of degree of flexion.

### DETAILED DESCRIPTION OF THE PREFERRED EMBODIMENT

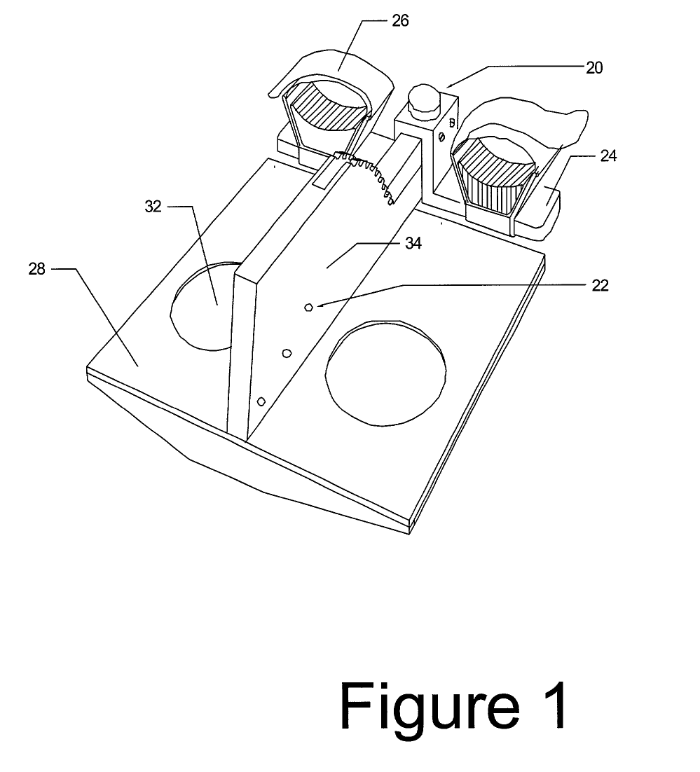

FIG. 1 is a perspective view of the appliance configured for imaging a knee in accord with the instant invention. All parts of the appliance consist of various plastics or rubbers chosen for the quality of transparency in the particular method of imaging. Under MRI imaging, these materials are generally anhydrous plastic resins and rubbers.

The cowling 34 is the principle structural component of the appliance. The base of the cowling 34 wraps around a central lever arm 20 to form a housing that extends longitudinally along the length of the enclosed lever arm 20. The cowling provides a fixation for rotational pivot point 22 as well as fixation points for the axels of pulleys 74 and 76.

The cowling extends to form a baseboard for fixation of the knees. Two holes 32 in the base of the cowling 28 serve as receptacles for the patellae of the knees under study. These holes 32 allow free movement of the patellar tissue and attendant muscles and ligaments while fixing the vertex of movement at the lever arm axle pivot point 22 rotatively fixed within the cowling.

The lever arm 20 fixes lower legs extending from a knee under study. To accomplish this fixation across joints of different proportions, the free sliding leg plate 24 is slidingly attached to the lever arm 20. Leg cradles 26, themselves slidingly attached to the sliding leg plate 24 to allow lateral movement while at the same time serving to restrain the legs and convey pressure resulting from tension against the anterior aspect of the legs.

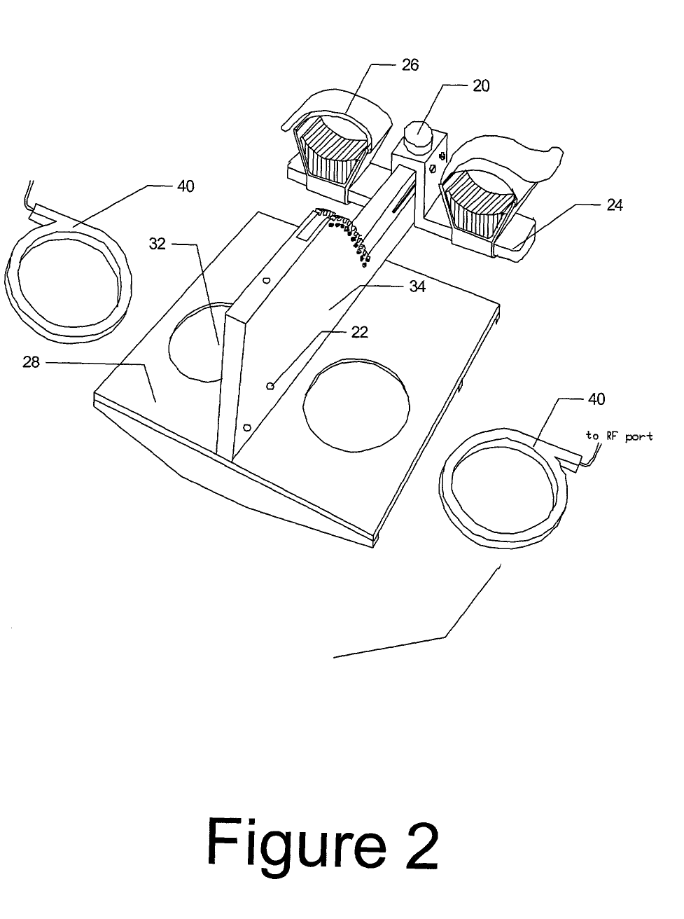

FIG. 2 shows electromagnetic coils 40 to refine the imaging of the knees under study. Positioning the coils below and concentrically with the holes 32 in the baseboard 28 allows intimate proximity to the knee joints under study. This proximity refines the imaging possible for viewing the ligaments the knee comprises.

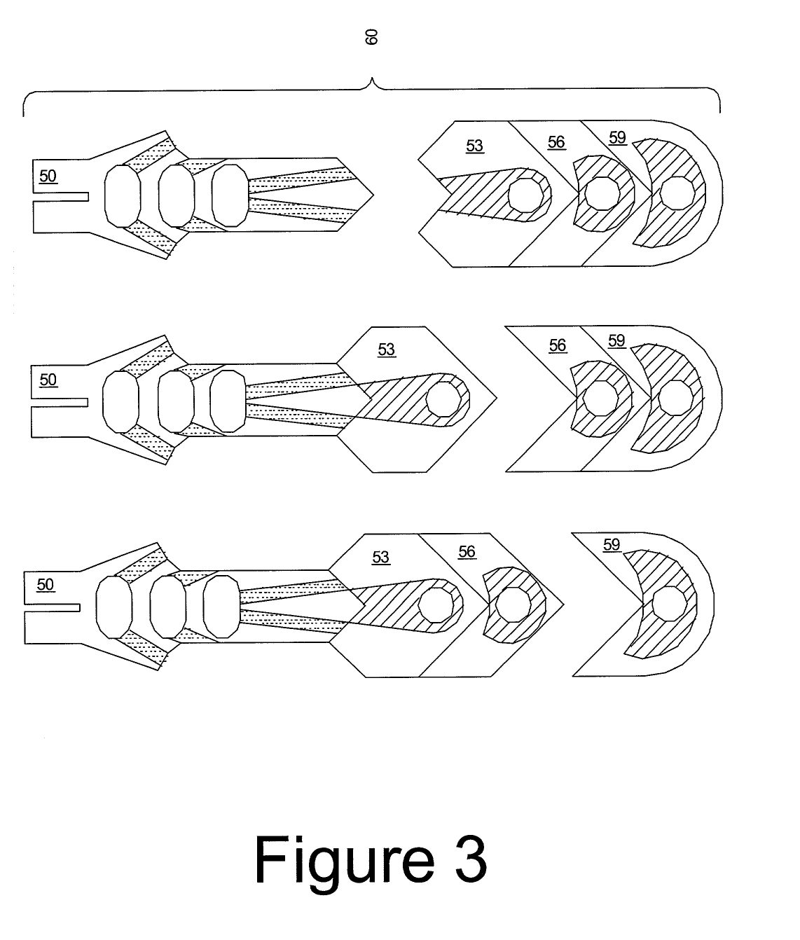

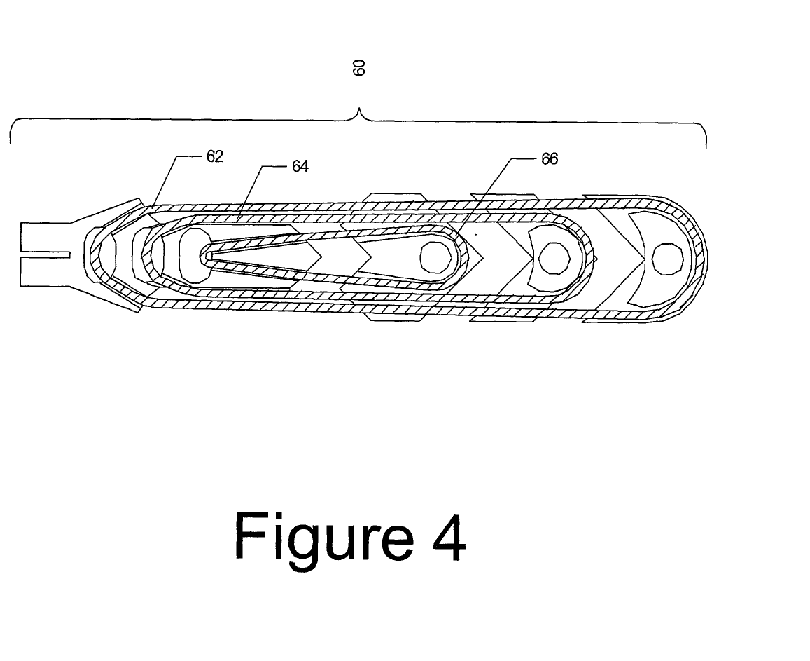

FIG. 3 shows one configuration of bushing blocks 53, 56, and 59 and an anchor block 50 together constituting elements of a variable tensioning device 60. Each of the bushing blocks bears a channel for receiving one of the several loops of elastic cord (not shown for clarity of illustration; see FIG. 4) used to present tension. The blocks move individually and each comprises a hole for fastening to the cowling 34. By fixing the tensioning device with the hole in block 56, for instance, an intermediate amount of pressure is applied to the knee. For more tension, the 53 block is used as indicated in the uppermost configuration in FIG. 3, for less, the 59 block as indicated in the lowermost configuration. The blocks mate and separate readily depending upon the fixation point.

FIG. 4 illustrates the whole tensioning unit 60, with the elastic loops 62 in place. Each loop consists of an elastomeric material that is selected for its transparency under examination. Block 50 comprises anchoring means for permanently affixing each loop, in turn, to the Block 50. So fixed, the loops are bedded and ready to receive the blocks 53, 56, and 59 to form the whole tensioning unit 60.

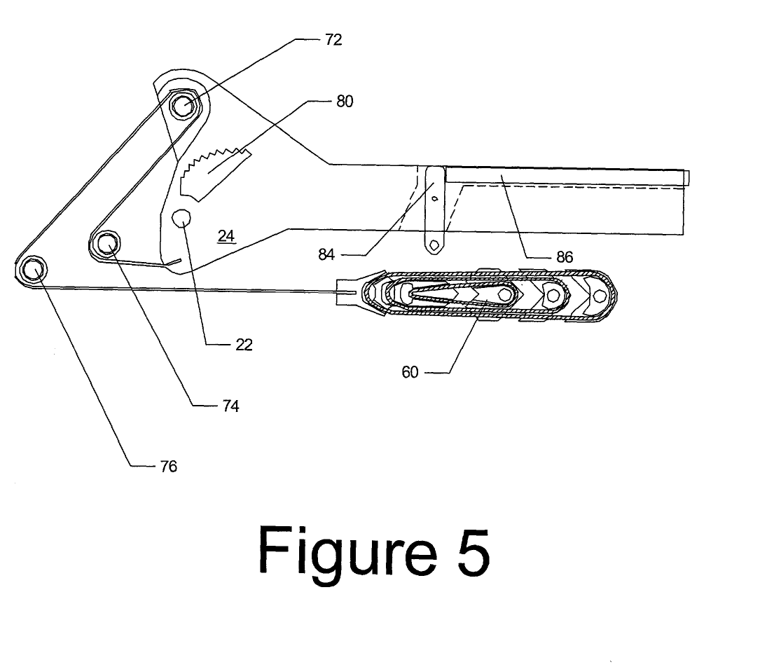

FIG. 5 illustrates the tensioning unit 60 in concert with the lever arm assembly 24. An inelastic cord 96 connects the tensioning unit 60 and is anchored to the control arm assembly 24. An inventive feature of the assembly is the configuration of the several pulleys both fixed on the cowling 74, 76, and the one on the lever arm 72. These pulleys are positioned in a manner to balance the increasing tension upon extension against the advantage gained by the pulleys 72, 74, and 76. Also indicated is the “phantom capsule” 80 (described in detail below) and the engaging pawl 84 and the disengaging button 86.

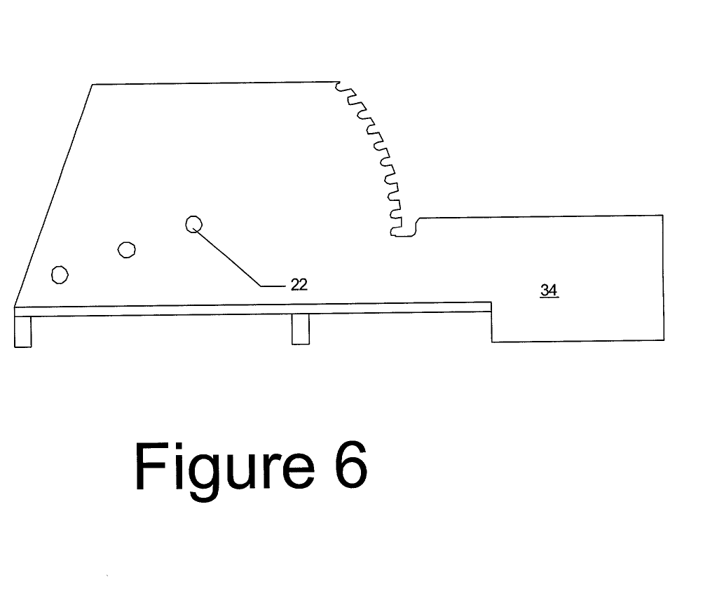

FIG. 6 illustrates a lateral cutaway view of the cowling that encloses the control arm assembly 24 portrayed in FIG. 5. The cowling comprises fixation points for the several pulleys and an axial pivot point 22. An arcuate segment with teeth is an additional feature of the cowling 34. The elastic attachment point within the cowling may be either a fixed or variable (variable by virtue of either a threaded rod attachment, or by a series of holes in the cowling sequential distal from pulley 22, or other means known in the art.

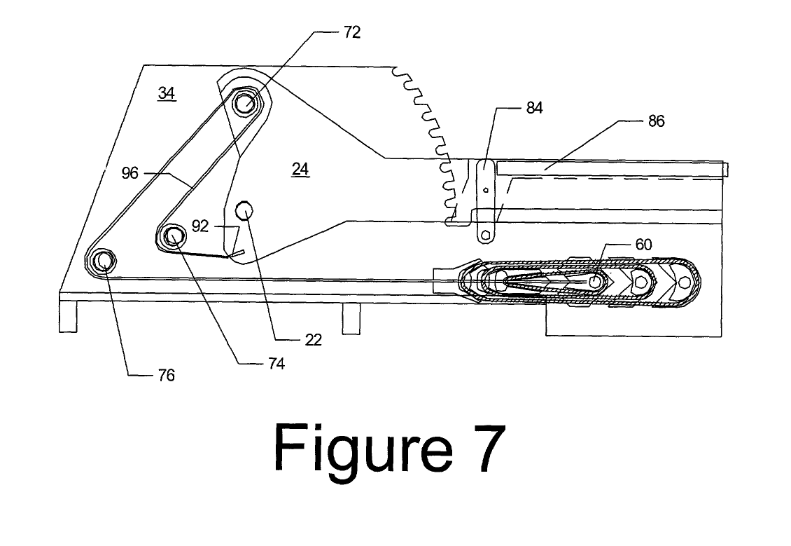

FIG. 7 portrays a simplified cutaway view of the lever arm 24 assembly within the cowling 34, thereby portraying the essential components of the lever system for placing pressure on the knee joint. As indicated above (portrayed at an angle of zero degrees of knee flexion), the pulleys 74 and 76 are pivotally attached to the cowling, while the pulley 72 and the anchor point 92 rotate around the pivot point 22. The tensioning unit 60 biases the cord 96 with a known tension.

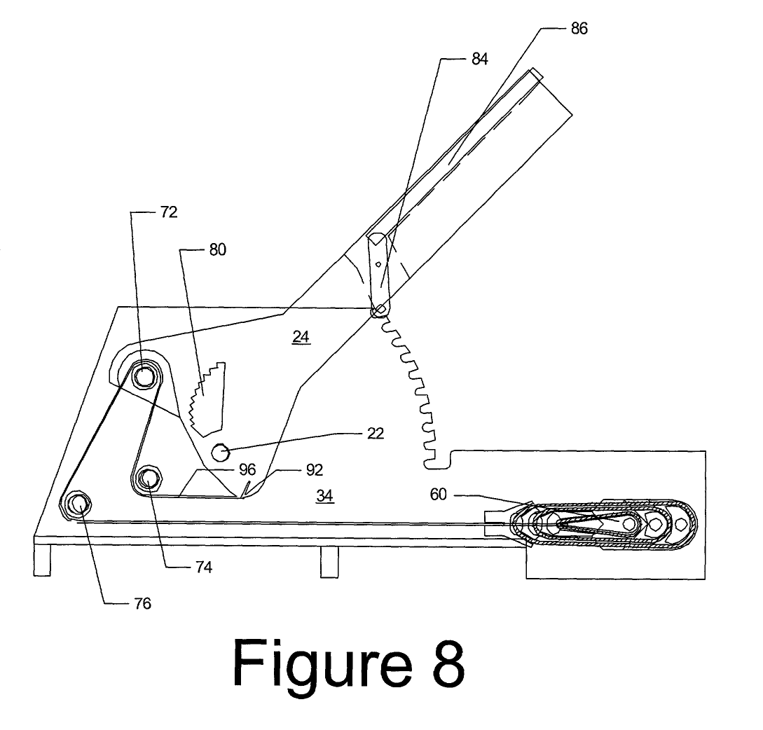

FIG. 8 portrays the cowling 34 and lever arm assembly 24 with the knee at 45° of flexion. Additionally portrayed are the “phantom window” 80 and the ratcheting pawl 84 engaged in the toothed arcuate surface of the cowling 34. As the knees are moved from 0° to 45° of flexion, the pawl 84 can alternately engage or disengage from the arcuate surface of the cowling 34 selectively providing a ratcheting of fixing of the knee in place when desired. Fixation is achieved by the interplay of tension applied through the cord 96 by the tensioning unit 60 and detent provided by the pawl 84. Simple pressure on the button 86 disengages the pawl 84.

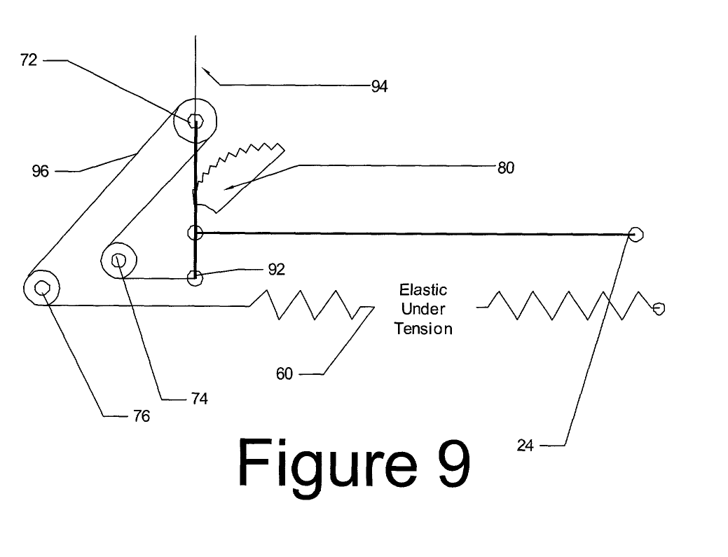

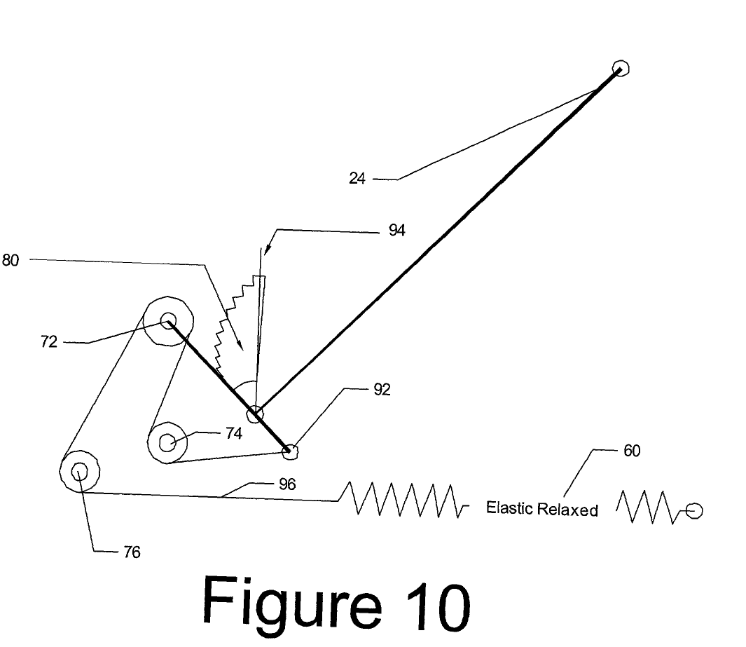

FIGS. 9 and 10 illustrate the geometry of the working of the lever arm assembly 24 as the lever arm moves from 0° to 45°. As discussed above, the geometry of the pulleys is selected to optimize the application of tension by interplay of the advantages imparted by the lever arm 24 and the several pulleys 72, 74, and 76. Generally, the configuration shown will impart uniform tension throughout the range of motion. The tensioning unit 60 is portrayed as a simple spring. The preferred embodiment, however, is shown in FIGS. 4-8.

Apparent in FIGS. 9 and 10 is the “phantom window” 80, a capsule or plastic vial containing an electrolyte solution such as an aqueous solution of manganese chloride, mixed to known concentrations in order to mimic the resonance of body tissue under the operation of the imaging apparatus. The purpose of the “phantom window” 80 is to impart to resulting image an indication of the degree of knee flexion portrayed. This the “phantom window” 80 does by producing an image of variable length.

When under examination, the center of the knees intersect and imaging plane. The imaging plane 94 intersects the “phantom window” capsule 80 radially from the lever arm axle 22. Movement of the lever arm 24 along an arc, pivoting at the lever arm axle 22 draws the “phantom window” capsule 80 across the imaging plane. Discrete steps in the wall of the “phantom window” capsule vary the silhouette of the capsule 80 intersecting the imaging plane 94 as the lever arm 24 moves.

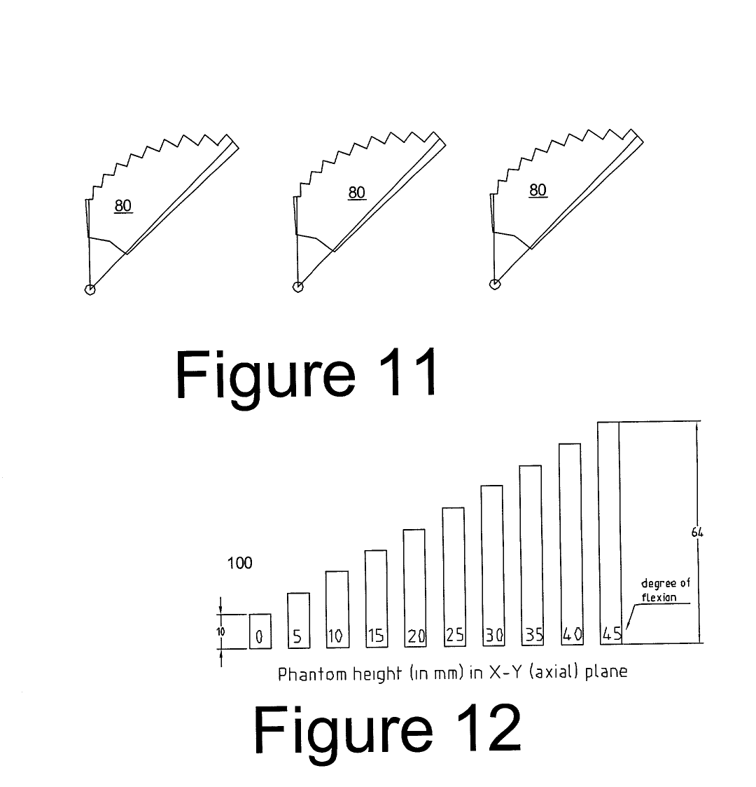

FIG. 11 portrays the “phantom window” capsule 80 as drawn across the imaging plane when the lever arm 24 reaches an angle of forty-five degrees of knee flexion showing the phantom capsule and its intersection with the imaging plane. FIG. 12 portrays a series of simplified images of the “phantom window” capsule at successive positions in a series of five degree increments from zero to forty-five degrees of knee flexion. The progressive lengths on the imaging plane are indicative of the flexion of the knee.

While the preferred embodiment of the invention has been illustrated and described, as noted above, many changes can be made without departing from the spirit and scope of the invention. For example, the tensioning unit might configured with resin springs or by leading the cord 96 off screen for other tensioning. Another example might include a framework rather than a cowling for fixation of the axis 22 of the lever arm 24 and the several pulleys 74, and 76. Accordingly, the scope of the invention is not limited by the disclosure of the preferred embodiment.

The embodiments of the invention in which an exclusive property or privilege is claimed are defined as follows:

1. A device for positioning a knee joint in a leg for examination during a diagnostic procedure, by reproducibly positioning a lower extremity of the leg, in a predetermined degree of flexion relative to an upper extremity of the leg, said device comprising:
a support member, said support member configured to allow the knees to flex while fixing their location with respect to the supporting member;
a lever arm secured in proximity to the radial end to the cradling means and pivotally at its central end to the support member;
a cradling means for securing the lower extremity of the leg in a fixed relationship to the lever arm; and
a biasing means to place a bias on the lower extremity of the leg tending toward positioning the lower extremity of the leg in a predetermined relationship to the upper extremity of the leg;

2. The device of claim 1, wherein the lever arm comprises means for fixing the lever arm in angular relationship to the supporting means.

3. The device of claim 2 wherein the means for fixing the lever arm in angular relationship to the supporting means is selectably engageable.

4. The device of claim 3 wherein the means for fixing the lever arm in angular relationship to the supporting means comprises a ratchet.

5. The device of claim 3 wherein the means for fixing the lever arm in angular relationship to the supporting means comprises a series of positioned holes and pin.

6. The device of claim 3 wherein the means for fixing the lever arm in angular relationship to the supporting means comprises a cam locking mechanism.

7. The device of claim 3 wherein the means for fixing the lever arm in angular relationship to the supporting means comprises a threaded rod and a block.

8. The device of claim 1 wherein the cradling means comprises a cradle and strap slidingly affixed to a crossbeam, itself slidingly affixed to the lever arm in proximity to the radial end.

9. The device of claim 1 wherein the cradling means comprises a clamping shell slidingly affixed to a crossbeam, itself slidingly affixed to the lever arm in proximity to the radial end.

10. The device of claim 1 wherein the lever arm comprises a means to indicate the lever arm's angular relationship to the supporting means.

11. The device of claim 10 wherein the means to indicate the lever arm's angular relationship to the supporting means comprises a capsule itself comprising an electrolytic solution visible to the imaging means.

12. The device of claim 11, wherein the biasing means comprises an elastic cord affixed to the lever arm.

13. The device of claim 12, wherein the biasing means additionally comprises a first set of at least one pulley affixed to the supporting means and wherein the elastic cord passes through the first set of pulleys.

14. The device of claim 13, wherein the biasing means additionally comprises a second set of at least one pulley affixed to the lever arm and wherein the elastic cord passes the second set of pulleys.

15. The device of claim 12 wherein the elastic cord comprises an inelastic member and an elastic member.

16. The device of claim 15 wherein the elastic member comprises a series of blocks and a corresponding series of loops of elastomeric cord.

17. A method of imaging one or two knees, each on a leg comprising a lower extremity and an upper extremity, by means of magnetic resonance (“MRI”) comprising:
fixing one or two knees to a supporting member, said member being transparent to MRI;
securing the lower extremity of each knee to a lever arm hingedly attached to the supporting member and transparent to MRI; and
biasing the lever arm as tending to a predetermined position.

18. The method of claim 17 additionally comprising generating an image indicative of an angular relationship between the supporting member and the lever arm.

19. The method of claim 17 wherein biasing comprises affixing an elastic cord to the lever arm and tensioning said cord.

20. The method of claim 19 wherein tensioning comprises affixing the elastic cord to the supporting member.
